(19) United States
(12) Patent Application Publication
(10) Pub. No.: US 2026/0315706 A1
(43) Pub. Date: Oct. 15, 2026

(54) SHIELDED NEEDLE UNIT WITH AXIAL MOUNTING FEATURE

(71) Applicant: Novo Nordisk A/S, Bagsvaerd (DK)

(72) Inventor: Nicolai Michael Villadsen, Oelstykke (DK)

(21) Appl. No.: 19/476,361

(22) PCT Filed: May 16, 2024
(86) PCT No.: PCT/EP2024/063553

§ 371 Date: Oct. 17, 2025

(30) Foreign Application Priority Data
May 23, 2023 (EP) 23174830.2

(51) Int. Cl. A61M 5/34; A61M 5/24; A61M 5/32

(52) U.S. Cl. CPC A61M 5/346; A61M 5/2429; A61M 5/3271

(57) ABSTRACT

A drug delivery device comprising a spring-driven drive member adapted to rotate piston rod to expel a dose from a cartridge. A controller is axially splined to the drive member and held against a housing stop surface. An actuator member is adapted to move the controller axially out of engagement with the stop, this allowing the drive member to be rotated by the drive spring. The controller in splined engagement with the drive member is rotated helically back to the start position and reengages the stop to thereby halt the drive member.

The present invention generally relates to a drug delivery device intended for subcutaneous introduction of a fluid drug formulation, the device comprising a drive spring adapted to expel one or more predetermined dose volumes of the fluid drug formulation.

### BACKGROUND OF THE INVENTION

In the disclosure of the present invention reference is mostly made to drug delivery devices intended for use by a patient for subcutaneous administration of a fluid drug formulation, e.g. in the treatment of diabetes by delivery of insulin or a GLP-1 type drug, or in the treatment of growth disorders by delivery of growth hormone, however, this is only exemplary uses of the present invention. In the present context the terms “pre-determined”, “pre-set” or “fixed” denote a dose size which has been determined during manufacturing of a given device and which subsequently cannot be adjusted by the user.

Spring driven drug delivery devices for so-called “automatic” injection of dose volumes of a liquid drug are well known in the art. Some of these drug delivery devices use a compression spring for driving out the dose volume and other use a torsion spring.

When injecting e.g. traditional types of insulin it is considered important that the user has a large range of different dose volumes available to choose between in order to inject exactly the necessary dose volume for the individual user for each injection. Such drug delivery devices are consequently provided with a dose setting mechanism by which the user incrementally can select the size of the dose volume to be injected, e.g. as disclosed in U.S. Pat. No. 6,899,699. The dose expelling in U.S. Pat. No. 6,899,699 is automatic and driven by a spring which can either be a compression spring or a torsion spring.

However, for other types of liquid drugs, e.g. GLP-1 type of drugs, it is possible to have a treatment regimen for a given patient in which the same dose volume is injected for each injection. This can be accomplished by having a single-use spring-driven drug delivery device which is able to expel one single dose volume having a predetermined and fixed dose size. The user thus performs the injection and discards the single use injection device once it has been used. This provides ease of use and assures that the correct amount of drug is expelled for each injection. The drive spring may be strained by the user prior to use, or the device may be delivered to the patient with a pre-strained drive spring, e.g. as disclosed in WO 2021/122200.

An alternative to using a plurality of such single-use fixed dose devices is to use a drug delivery device adapted to expel a plurality of predetermined and equally sized dose volumes. Such drug delivery devices are sometimes referred to as multi-use fixed dose drug delivery devices. Examples of such multi-use fixed dose device for expelling a plurality of predetermined and equally sized dose volumes are disclosed in WO 2017/098460, WO 2018/007259 and in WO 2020/089167, the devices comprising a drive spring which is strained by the user prior to expelling a pre-set dose of drug. As an alternative, WO 2021/122219 discloses a multi-use fixed dose drug delivery device comprising a drive spring which come pre-strained for the plurality of doses.

US 2020/0261656 discloses a variable-dose drug delivery device comprising a clutch member which via a threaded connection can be rotated by axial movement of a dose tracker. The clutch member engages a piston rod drive member via a ratchet interface allowing the two members to rotate relative to each other during dose setting.

Having regard to the above, it is an object of the present invention to provide a drug delivery device of the fixed dose type comprising a drive spring, which can be manufactured cost-effectively, and which is both easy and safe to handle during operation and use. Such a drug delivery device may be adapted to expel a single or a plurality of fixed doses. It may be provided with a factory pre-strained drive spring, or the user may strain the spring for one or more doses prior to use.

### DISCLOSURE OF THE INVENTION

In the disclosure of the present invention, embodiments and aspects will be described which will address one or more of the above objects or which will address objects apparent from the below disclosure as well as from the description of exemplary embodiments.

In a first aspect of the invention a drug delivery device is provided, comprising or adapted to receive a drug-filled cartridge. The drug delivery device comprises a housing and an expelling assembly. The expelling assembly comprising a threaded piston rod adapted to engage and axially displace a piston in a loaded cartridge in a distal direction when rotated to thereby expel a dose of drug from the cartridge, a drive member adapted to rotate the piston rod and defining a reference axis, a drive spring coupled to the drive member, control means adapted to control a predetermined amount of rotation of the drive member, and actuatable release means adapted to release the control means to allow the drive spring to rotate the drive member the predetermined amount to thereby expel a predetermined dose amount. The control means comprises one or more axially oriented and circumferentially spaced apart stop surfaces, a control member in axially splined engagement with a portion of the drive member, the control member comprising an axially oriented control surface adapted to engage a stop surface, and axial control means adapted to move the control member in the distal direction as it is rotated by the drive member. The control member is axially moveable by the release means between a distal position in which the control surface engages a stop surface and thus prevents rotation of the drive member, and a proximal release position in which the control surface has disengaged the stop surface, this allowing the drive member to be rotated by the drive spring. The control member is moved distally by the axial control means until the combined axial and rotational movement of the control member causes the control surface to engage a stop surface and thereby stop rotation of the drive member, the predetermined dose size being determined by the circumferential spacing of the respective stop surfaces.

By this arrangement rotational control of the drive member is provided assuring that the expelled dose size is determined by the physical arrangement of the control respectively the stop surfaces. Further, as only the control member is moved axially an axially compact drug delivery device is provided.

The axial control means may be in the form of a helical follower arrangement providing that rotational movement of the control member is transformed to axial movement of the control member. In a helical follower arrangement at least one helical structure engaging at least one follower structure assures that rotation of one structure is transformed to axial movement of the other structure when the two structures are held in engagement. Engagement may be provided by a threaded arrangement in which opposed surfaces of the helical structure is engaged by a pair of followers. For example, the followers may in the form of corresponding helical surfaces as in a traditional threaded connection. If only one follower structure is provided engagement may be provided by a spring urging the helical structure and the follower into axial engagement with each other.

In an exemplary embodiment the helical follower arrangement comprises a thread portion, e.g. a helical flange portion, on the control member, the thread portion having a distally facing surface adapted to engage a distal thread structure. The thread portion may also have a proximally facing surface adapted to engage a proximal thread structure to thereby provide a threaded connection adapted to move the control member distally as it is rotated. The distal thread structure may be provided by a portion of the housing, e.g. in the form of a proximally facing helical surface portion, and the proximal thread structure may be provided by a portion of the release member, e.g. a distally facing surface portion on the release member.

In an exemplary embodiment the release means comprises a release member adapted to engage the control member, the release member being axially moveable between a non-engaged distal position and a proximal release position in which the control member has been moved to its proximal release position. A return spring may be provided for biasing the release member in the distal direction.

When a proximal thread structure is provided it may disengage the control member thread portion when the predetermined dose size has been expelled, e.g. by a cut-out in the thread structure, this allowing the return spring to move the release member in the distal direction.

In an exemplary embodiment a single axially oriented stop surface and a single axially oriented control surface is provided, whereby the control member rotates 360 degrees between the control stop disengaging and re-engaging the stop surface. Alternatively, if two stop respectively two control surfaces were provided the control member would rotate 180 degrees between the control stop disengaging and re-engaging the stop surface.

The drive spring may be a torque drive spring engaging the drive member respectively the housing (900, 932), the drive spring may be pre-strained corresponding to a predetermined number of doses. Alternatively, it may be strained by the user before each dose expelling.

The drug delivery device may be provided in combination with a needle unit, the drug delivery device comprising a cartridge mount allowing the needle unit to be arranged in fluid communication with the cartridge interior. The needle unit comprises a needle hub, a hollow needle mounted in the needle hub and having a pointed distal end protruding from the needle hub, and a shield in which the needle hub is arranged, the shield being axially moveable relative to the needle hub between an extended position in which the shield axially covers the needle distal end, and a retracted position in which the needle distal end protrudes from the shield. A hub coupling allows the needle unit to be mounted on the drug delivery device cartridge mount. When the needle unit is mounted on the cartridge mount, the release means, e.g. an axially moveable actuator member, is actuated to release the control means when the shield is moved from the extended to the retracted position. Correspondingly, a return spring acting on the actuator member can serve to return the shield to its extended position covering the needle distal end when the needle is withdrawn from the user's skin.

As used herein, the term “drug” is meant to encompass any drug-containing flowable medicine capable of being passed through a delivery means such as a hypodermic needle in a controlled manner, such as a liquid, solution, gel or fine suspension. The drug may have a blood glucose controlling effect, e.g. human insulin and analogues thereof as well as non-insulins such as GLP-1 and analogues thereof.

### BRIEF DESCRIPTION OF THE DRAWINGS

In the following embodiments of the invention will be described with reference to the drawings, wherein:

FIG. 1 shows a drug delivery device with a mounted needle unit,

FIG. 2 shows in a cross-sectional view the drug delivery device 1 with the needle unit being replaced with a cap,

FIG. 3 shows an exploded view of the components of the drug delivery assembly of FIGS. 1 and 2,

FIGS. 4A and 4B show a perspective respectively a cross-sectional view of the cartridge holder of FIG. 3,

FIGS. 5A and 5B show a perspective respectively a cross-sectional view of the drive nut of FIG. 3,

FIGS. 6A-6C show a perspective respectively cross-sectional views of the housing member of FIG. 3,

FIGS. 6D and 6E show in cut-away views details of the structures surrounding the housing tower,

FIGS. 7A and 7B show a perspective respectively a cross-sectional view of the actuator of FIG. 3,

FIG. 8 shows a perspective view of the return spring of FIG. 3,

FIGS. 9A and 9B show a perspective respectively a cross-sectional view of the control member of FIG. 3,

FIGS. 10A and 10B show a perspective respectively a cross-sectional view of the drive member of FIG. 3,

FIG. 11 shows a perspective view of the piston rod of FIG. 3,

FIG. 12 shows a perspective view of the drive spring of FIG. 3,

FIGS. 13A and 13B show a perspective respectively a cross-sectional view of the spring base member of FIG. 3,

FIG. 14 shows in cross-section the drug delivery device of FIG. 2, the cap, the cartridge, the cartridge holder and the return spring being removed for better visibility,

FIG. 15 shows a detail proximal cross-sectional view of the drive spring arranged in the spring base,

FIG. 16 shows a detail distal cross-sectional view of the drive spring arranged in the spring base,

FIG. 17 shows a detail cross-sectional view of an actuator drop lock flexible finger in engagement with a housing drop lock stop surface,

FIGS. 18A-18F show in a series of perspective views movement of the actuator knob and control member relative to the housing tower portion during expelling of a dose of drug,

FIGS. 19A and 19B show a perspective respectively a cross-sectional view of the shield member of FIG. 3,

FIGS. 20A and 20B show a perspective respectively a cross-sectional view of the hub member of FIG. 3,

FIGS. 21A and 21B show a perspective respectively a cross-sectional view of a container for the needle unit of FIG. 1,

FIG. 22 shows a cross-sectional view of the needle unit of FIG. 1 arranged in the container of FIG. 21A, and

FIGS. 23A-23C show in a series of cut-away perspective views movement of the shield and needle hub during actuation.

In the figures like structures are mainly identified by like reference numerals.

### DESCRIPTION OF EXEMPLARY EMBODIMENTS

When in the following terms such as “upper” and “lower”, “right” and “left”, “horizontal” and “vertical” or similar relative expressions are used, these only refer to the appended figures and not necessarily to an actual situation of use. The term “distal” refers to a portion of an element, assembly or device which during use is oriented towards a skin surface of a user, the term “proximal” referring to the opposed portion. The terms “axially” or “axial orientation” refer to a direction in parallel with a general reference axis. Correspondingly, for a traditional pen drug delivery device the needle is arranged at the distal end and an end-mounted release button is arranged at the proximal end. The shown figures are schematic representations for which reason the configuration of the different structures as well as their relative dimensions are intended to serve illustrative purposes only. When the term member or element is used for a given component it generally indicates that in the described embodiment the component is a unitary component, however, the same member or element may alternatively comprise a number of sub-components just as two or more of the described components could be provided as unitary components, e.g. manufactured as a single injection moulded part. The term “assembly” does not imply that the described components necessarily can be assembled to provide a unitary or functional assembly during a given assembly procedure but is merely used to describe components grouped together as being functionally more closely related.

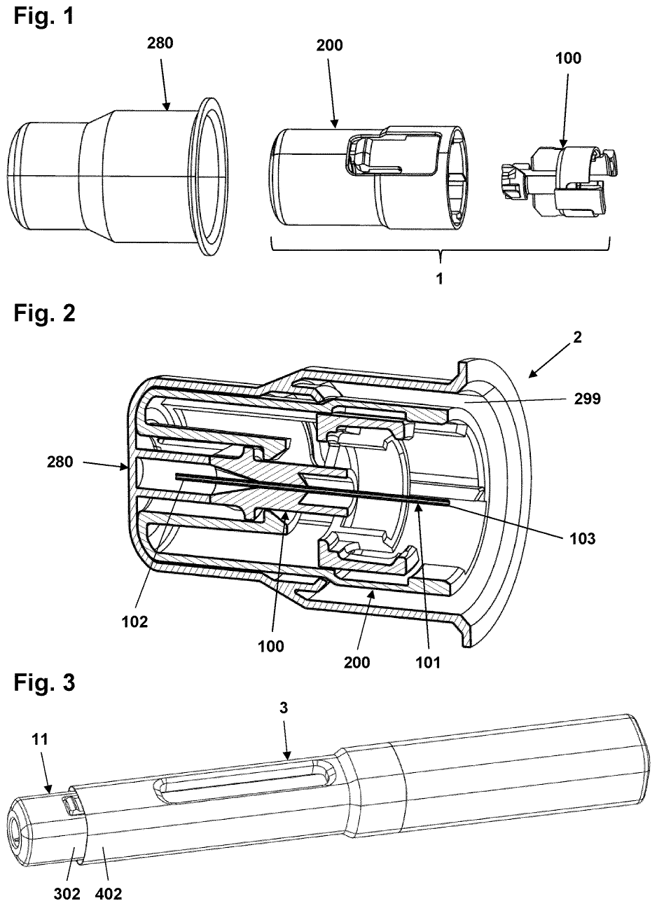

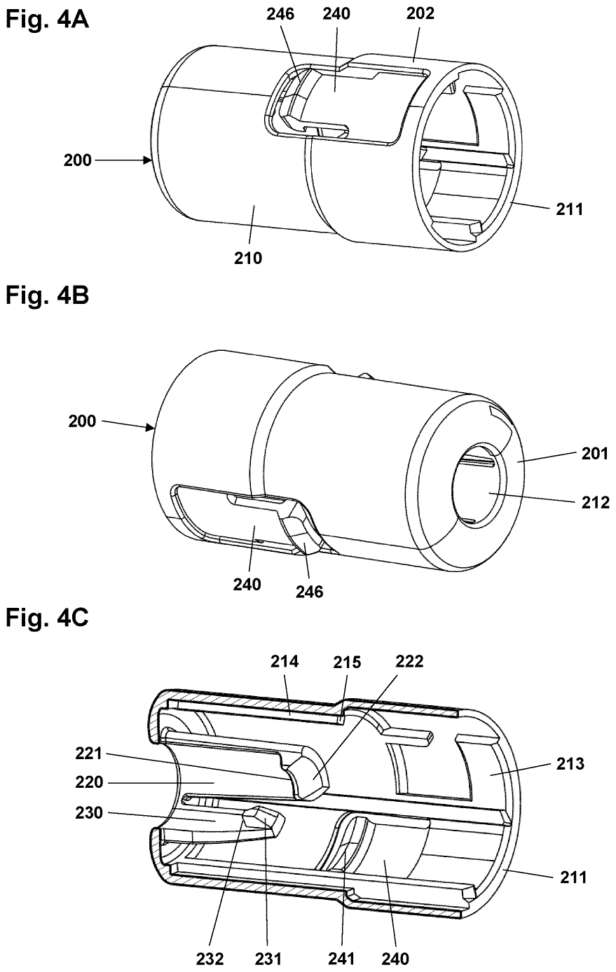

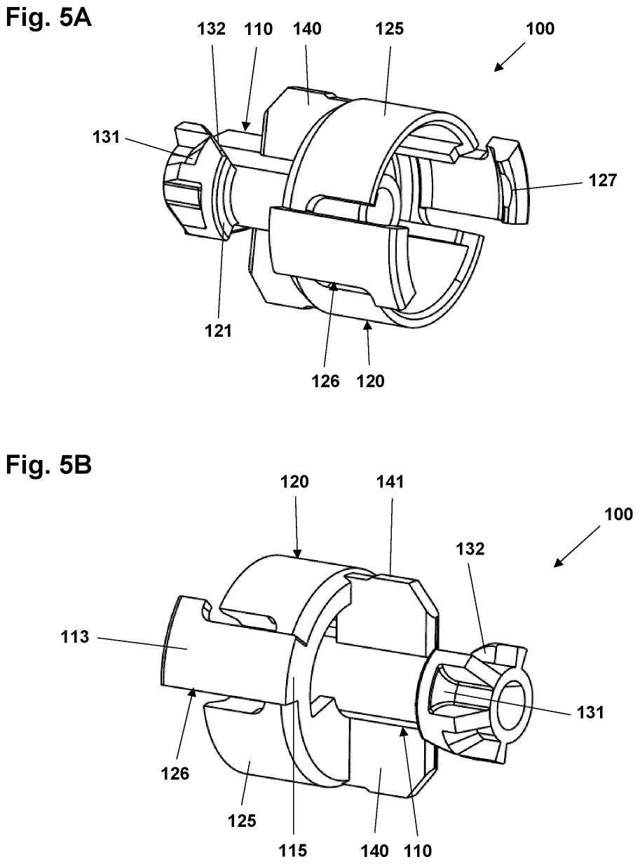

With reference to FIG. 1 a drug delivery device 1 with a mounted needle unit 2 is shown. The device has a general tubular configuration defining a general reference axis. FIG. 2 shows in a cross-sectional view the drug delivery device 1 with the needle unit being replaced with a cap. In the shown embodiment the cap fits snugly over the distal part of the drug delivery device and thus does allow a needle unit to be mounted at the same time. The drug delivery device comprises a distal cartridge holder portion 3 in which a drug filled cartridge is arranged, a proximal portion comprising a drive spring system 4 as well as an intermediate portion comprising a control system 5. A piston rod is arranged axially in the device and adapted to be moved distally by the drive spring to expel an amount of fluid drug through a mounted needle unit, the amount of axial travel of the piston rod being controlled by the control system. In FIG. 2 the actuator return spring is not shown.

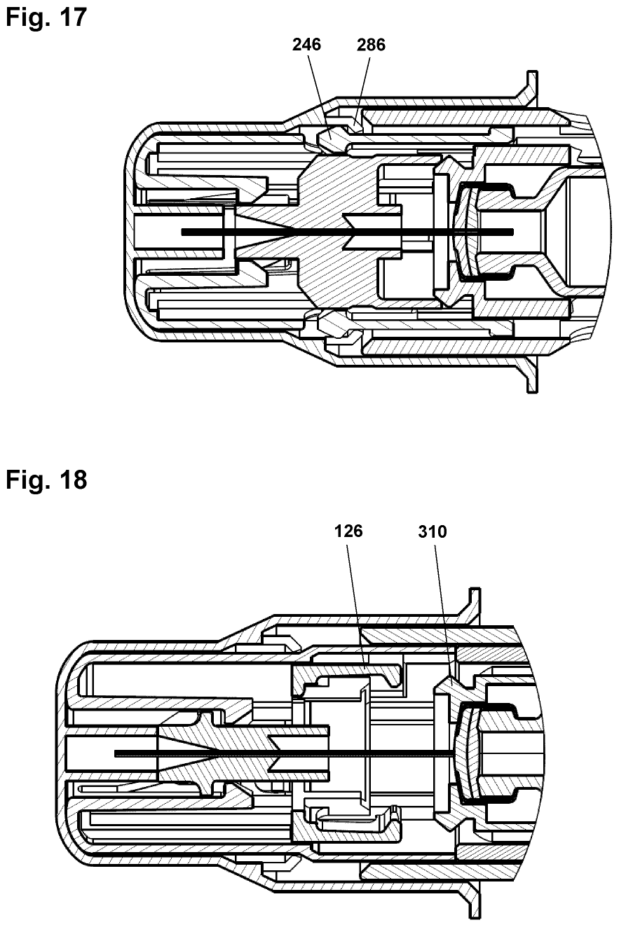

In the exploded view of FIG. 3 the individual components of the drug delivery device and needle unit are shown. The drug delivery device comprises a tubular housing 400 with a proximal engine portion 401 and a distal cartridge portion 402 adapted to house a cartridge holder 300 in which a drug cartridge 390 is arranged, the drug cartridge comprising a distal outlet end, a proximal circumferential edge 391 and an axially displaceable piston 392 (see FIG. 17). A piston washer 395 is arranged in the cartridge engaging the piston proximal surface. An actuator 500 comprises a cylindrical proximal portion 501 from which a pair of legs 502 extend distally interposed between the housing and the cartridge holder. The drive nut 600 is mounted in the housing and adapted to receive piston rod 650 in threaded engagement. The piston rod is non-rotationally received in the distal tubular portion 802 of drive member 800 which is adapted to be rotationally driven by pre-strained drive spring 890 arranged in the proximal portion 801 of the drive member, the drive spring's proximal end being anchored to the housing via spring base 900. Control member 700 is in splined engagement with the drive member tubular portion 802 and is adapted to be moved axially in and out of engagement with the housing to thereby control rotation of the drive member. A large-diameter return spring 590 is arranged to provide a distally directed biasing force on the actuator. The drug delivery device is adapted to receive a needle unit at its distal end, the needle unit comprising a needle hub 100 with a needle 101, the needle hub being arranged in a shield member 200. When a needle unit is not mounted on the drug delivery device a cap 490 can be mounted to cover the cartridge portion 402. In the following the individual components will be described in greater detail. The needle unit will be described after the drug delivery device.

The cartridge holder 300 has a generally cylindrical configuration with a larger proximal opening 301 allowing a cartridge to be inserted and a smaller distal opening allowing a hollow needle to be inserted through a septum of a mounted cartridge. The distal end comprises a cartridge mount 310 with a pair of opposed part-circumferential flange portions 311 adapted to receive a pair of flexible hub coupling arms (see below). Neighbouring the flange portions are portions 312 with lower and/or less inclined release flanges allowing the hub coupling arms to disengage with ease. The cylindrical main portion comprises a pair of opposed longitudinal openings 315 allowing a user to inspect the content of the cartridge as well as the position of the cartridge piston. Each opening is surrounded by a radially extending window wall 316 adapted to engage with a corresponding window 416 in the housing 400, the wall comprising a number of indicator lines 317 arranged to align with the cartridge piston during operation. The cartridge proximal end comprises a pair of opposed circumferentially extending flexible support arms 320 each having a free end portion with a protrusion 321 extending radially into the opening 301, the flexibility of the arm allowing the protrusion to be moved radially out of the opening to allow insertion of a cartridge. A free space 322 is provided distally of the flexible support arm allowing the support arm to be deformed into the free space when engaging the housing pressure edges 437 during assembling to thereby eliminate axial play of a mounted cartridge (see below). The flexible arms together with the neighbouring parts of the cartridge holder form a cartridge holder proximal edge 323 with gaps corresponding to the flexible arm free ends. In the shown embodiment the edge is arranged generally in a transversal plane.

The drive nut 600 comprises a generally tubular main portion 610 with an interior thread 611 adapted to engage a corresponding thread on the piston rod 650. The drive nut comprises a first pair of opposed distally oriented flexible mounting fingers 620 adapted to be received in corresponding nut slots in the housing central portion. Each finger is provided with a small flexible finger 621 allowing the drive nut to snap into engagement with the housing—at this stage with a small axial play. The fingers are oriented slightly outwards to secure proper contact with the nut slots during subsequent fixation of the drive nut in the housing, e.g. by application of laser energy through an opening in the housing (see below). The drive nut further comprises a second pair of opposed circumferentially extending flexible fingers 630 each being inclined in the proximal direction to provide a distally directed biasing force during assembling.

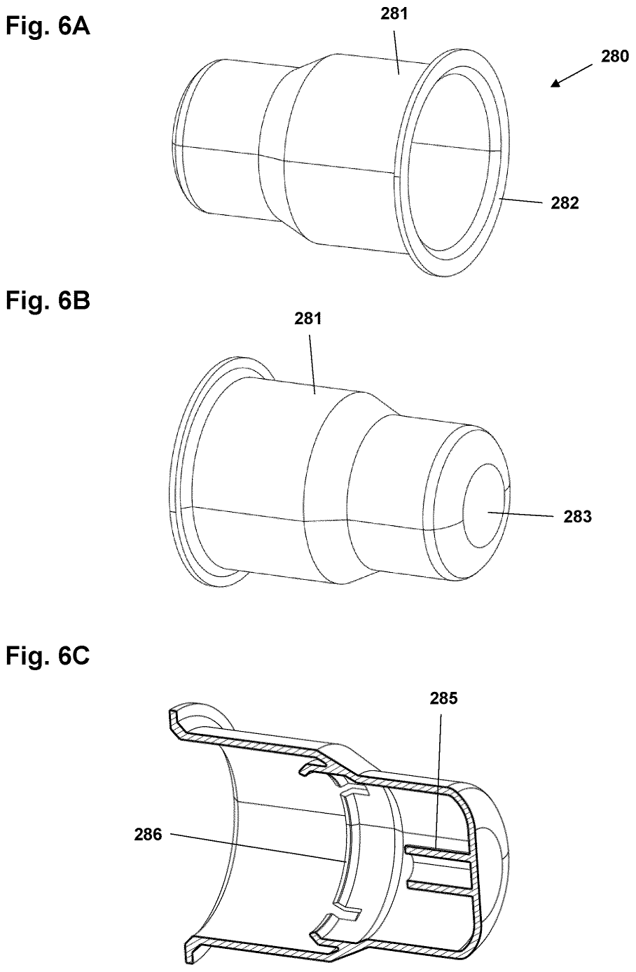

As shown in FIGS. 6A, 6B and 6C the generally tubular housing 400 comprises a proximal engine portion 401, a distal cartridge portion 402 and a generally cylindrical tower portion 430 arranged distally in the engine portion. In the shown embodiment the distal and proximal portions both have a super-elliptic cross-section with n=2.5 and a≠b. The tower distal portion is connected to the outer housing via a pair of opposed bridge portions 440 forming a pair of openings therebetween providing axial passageways for the actuator legs 502 (see below) between the cartridge and engine portions. Each bridge portion 440 is provided with a proximally extending lock rib 447 adapted to engage the actuator triangular index positions (see below). A neighbouring proximally facing rib 448 serves as a support for the actuator. The cartridge portion 402 comprises a distal indicator opening 405, a pair of opposed windows 410, and a distal pair of opposed shield slots 415 adapted to receive corresponding shield mounting ribs 245 (see below). Proximally on the inner surface a pair of opposed inwardly protruding and distally facing drop lock stop surfaces 407 are provided, as well as a pair of opposed proximally extending point-of-no-return (PONR) flexible arms 408 with a proximally facing PONR stop surface 409 (see FIG. 6D). On the outer surface a pair of opposed snap protrusions 403 adapted to engage the cap 490 is provided. The engine portion 401 comprises a distal pair of opposed nut openings 406 allowing access to the drive nut mounting points during assembly, e.g. allowing the drive nut to be fixed to the housing using a laser beam, as well as a proximal pair of opposed flexible snap lock fingers 420 allowing the spring base 900 to be mounted fixedly in the housing and thus form part of the housing, the snap lock fingers each comprising an inwardly protruding snap hook 421. A number of inner axial ribs 404 provide radial support for the actuator. The tower comprises a pair of opposed axially oriented inner nut slots 432 adapted to receive the drive nut mounting fingers 620. The distal edge of the tower forms a pair of opposed part-circumferential support flanges 431 adapted to be received in the cartridge holder proximal opening 301. Each support flange is associated with a radially extending pressure edge 437 (see FIG. 6E) adapted to engage and deform the cartridge holder flexible arm when the cartridge holder is inserted in the housing (see below). The tower further comprises a proximally oriented helical control surface 433, the ends of the helical surface being connected with an axially oriented stop surface 434 which extends distally to create a notch 435 in the helical surface. Centrally in the tower an inner circumferential stop flange 438 is provided adapted to engage the drive member 800. Proximally of the stop flange a stop knob 439 is located serving as a second rotational stop for the control member. In the shown embodiment a single indicator opening 405 is provided but alternatively a second opposed opening could be provided.

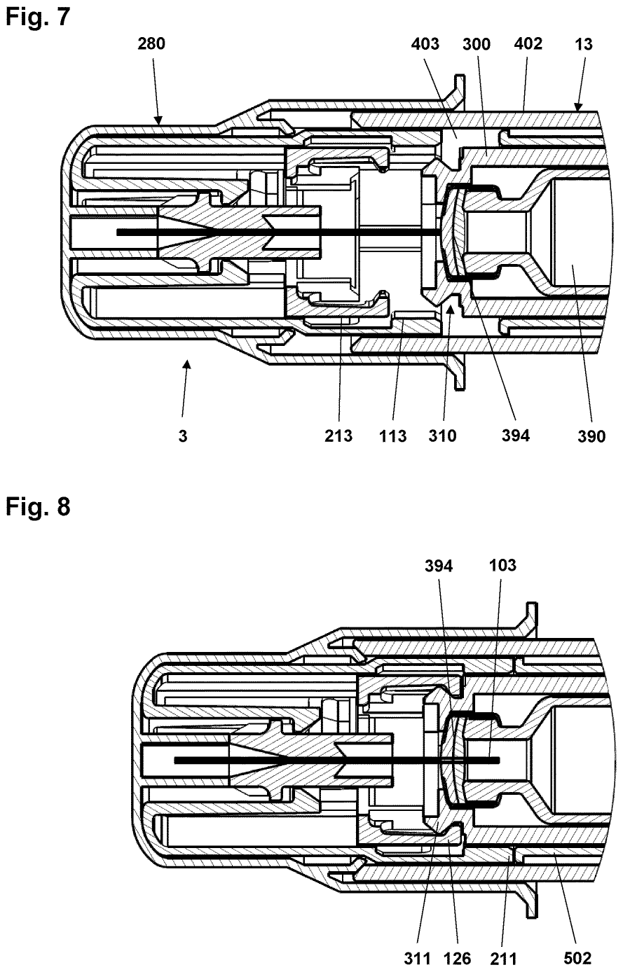

As shown in FIGS. 7A and 7B the actuator 500 comprises a cylindrical proximal portion 501 from which a pair of legs 502 extend distally. The cylinder portion comprises a distal outer circumferential spring support flange 510 adapted to engage return spring 590, as well as an inner actuator knob 516 and drop lock flange 511, the latter providing an axial stop for the control member helical flange 710 (see below) and thus serves as a drop lock for the control member 700. The cylinder portion distal edge is provided with a pair of serrated clutch portions 517 each creating two triangular index positions adapted to engage the housing lock ribs 447 in an initial respectively in an actuated rotational position, thereby providing an anti-rotational clutch when engaged. Each actuator leg 501 comprises a proximal outwardly protruding flexible drop lock finger 520 with a proximally facing drop lock surface 521 at the proximal free end adapted to engage a housing drop lock stop surface 407. Each actuator leg 501 further comprises a proximal axially oriented PONR wedge structure 530 with a distally facing stop surface 531 adapted to engage a PONR proximally facing housing stop surface 409. At the distal end each actuator leg comprises a distal-most actuation surface 523 adapted to engage the needle shield 200 as well as a neighbouring inclined release surface 524 adapted to engage the needle hub 100.

The helical return spring 590 shown in FIG. 8 is generally open wound but to prevent tangling during manufacturing the spring is provided with distal, central and proximal tightly wound sections.

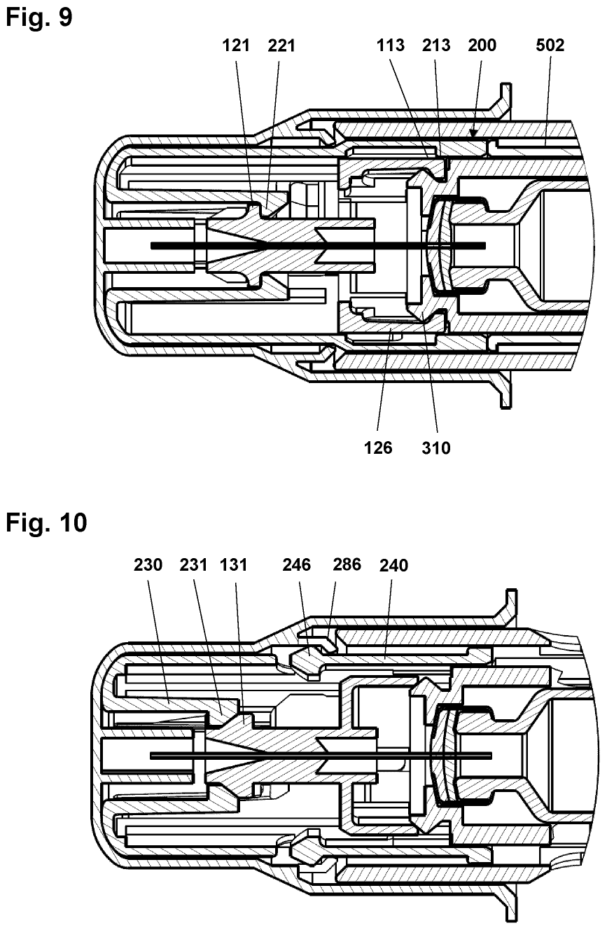

As shown in FIGS. 9A and 9B the generally tubular control member 700 comprises an outer helical flange 710 serving to control axial movement of the control member relative to the housing. The helical flange comprises a helical distal surface 713 adapted to engage the helical surface 433 on the tower, a helical proximal surface 719 adapted to engage the distal end of the actuation knob 516, and a longitudinally extending stop edge 714 connecting the ends of the helical flange 710 and being adapted to engage the stop edge 434 on the housing tower. Rotationally aligned with the control member stop edge 714 an axially oriented release flange 716 is arranged on the outer surface of the control member. In the distal direction the stop edge 714 extends to form a pointed catch 715 adapted to be seated the tower notch 435. The majority of the helical flange 710 has a first larger diameter whereas cut-out portions 711 on each side of the stop edge 714 have a smaller diameter corresponding to the height of the stop edge, this allowing the actuator knob 516 to axially pass. The control member further comprises a distally oriented helical edge 720, the ends of the helical edge being connected with an axially oriented second stop edge 729 adapted to engage tower stop knob 439. The control member inner surface comprises an opposed pair of spline ridges 730 adapted to engage corresponding spline slots in the drive tube. At the distal end and aligned with the spline ridges an opposed pair of stop flanges 735 are arranged adapted to engage the piston rod stop surface 655.

As shown in FIGS. 10A and 10B the drive member 800 comprises a proximal cylindrical spring housing portion 801 and a distal drive tube portion 802. The drive tube portion comprises at the distal end an inner pair of opposed axially oriented drive flanges 810 adapted to engage the piston rod drive grooves. Along the length of the drive tube portion a pair of opposed spline slots 830 is provided adapted to engage the corresponding control member spline ridges 730. As described below, the spline slots may comprise friction structures providing that the control member cannot slide freely on the drive member 802. The spring housing 801 comprises at the distal end an inner circumferential flange creating a circumferential groove 820 adapted to receive the distal end of the drive spring, the groove comprising an oblong catch opening 821 adapted to receive the drive spring distal hook. The spring housing comprises an opposed pair of flexible arms 822 protruding slightly into the interior of the spring housing, the arms being adapted to engage and axially hold the spring in place during assembling. At the proximal end an outer part-circumferential flange 823 is provided adapted to support the return spring 590, as well as a single circumferentially oriented and outwardly protruding flexible clicker finger 825 having a free end adapted to rotationally engage a circumferential ratchet surface 925 on the spring base 900.

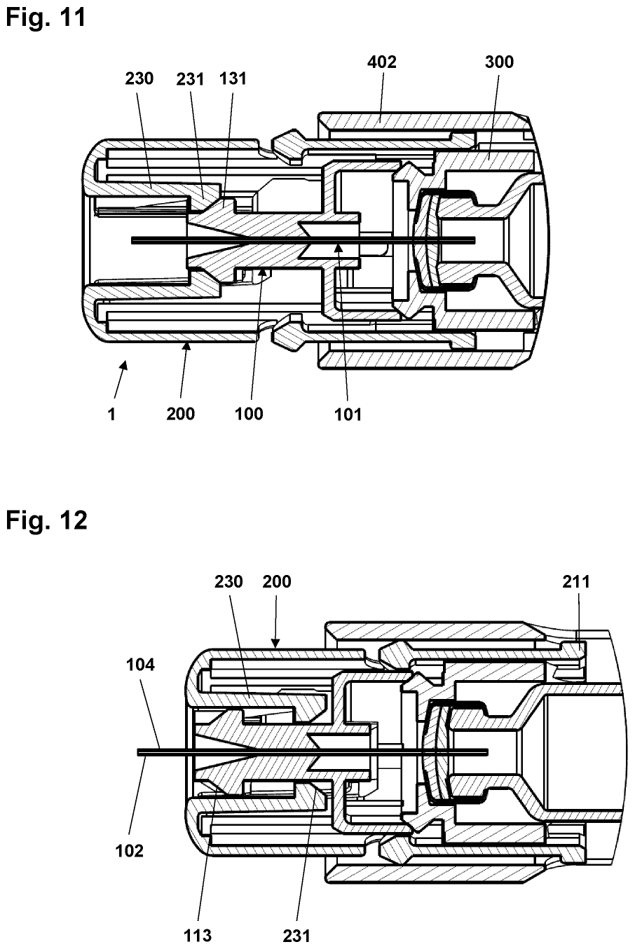

FIG. 11 shows the piston rod 650 comprising along its length a thread 651 as well as a pair of opposed drive grooves 652. The proximal end comprises a distally facing stop flange 655 adapted to engage the control member stop flanges 735. The distal end 654 is adapted to engage the piston washer 395 which in the shown embodiment is a flat disk. Alternatively the piston washer may be provided with proximally facing centring means adapted to engage the piston rod distal end, e.g. the piston washer and the piston rod distal end may be provided with corresponding snap coupling structures.

The drive spring 890 as shown in FIG. 12 is a tight wound helical spring without any spacing between adjacent coils and comprises a distal hook portion 891 arranged in an axial plane and a proximal hook portion 892 arranged in a transversal plane.

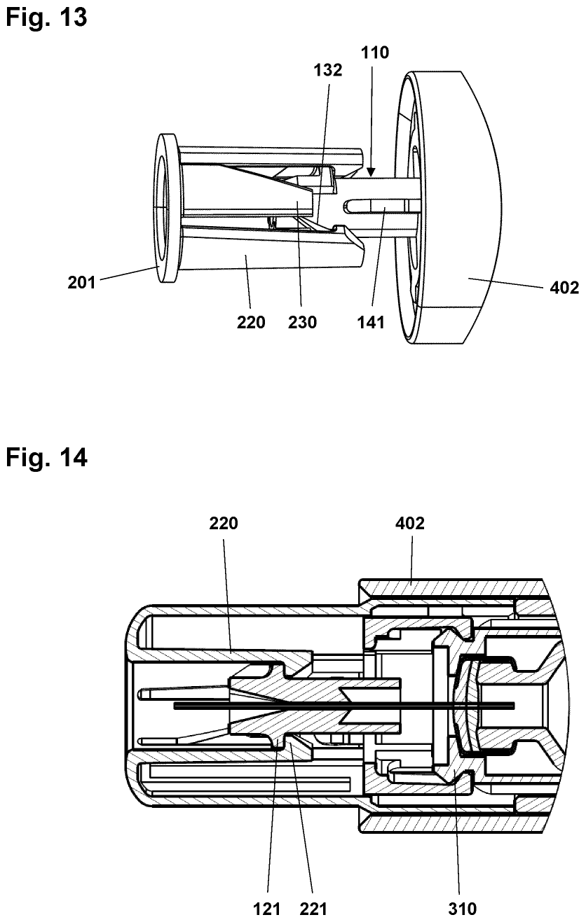

As shown in FIGS. 13A and 13B the spring base 900 comprises a proximal planar end surface 901 from which extend in the distal direction an outer circumferential skirt 902, an inner circumferential skirt 910 and a central tower portion 920 providing an inner support for the drive spring. Distally the inner surface of the inner skirt comprises a circumferential ratchet surface 915 adapted to engage clicker finger 825. The inner skirt further comprises an opposed pair of catch openings 911 adapted to engage the housing snap lock finger hooks 421. Between the inner skirt and the central tower portion an opposed pair of distally extending fingers 930 are provided, each finger comprising a longitudinally extending free spring edge 932 adapted to engage the drive spring proximal hook 892 in axial sliding engagement. The sliding engagement allows the spring base to be inserted into the housing after the spring has been strained just like the spring hook can slide on the spring edge as the spring shortens during de-straining.

Having described the individual components of the drug delivery device 1, FIG. 14 shows a cross-sectional view of the assembled device. For clarity the cartridge holder, the cartridge and the return spring has been removed, however, the former components can be seen in FIG. 2. More specifically, the strained drive spring 890 is arranged in the spring housing 801 providing a rotational force to the piston rod 650 via the drive tube 802 and thereby distal axial movement through the treaded engagement with the drive nut 600. The drive tube is in splined engagement with the control member 700 and thus prevented from rotating as long as the control member is in its rotationally “parked” position with the control member stop edge engaging the tower stop edge (see below). The control member is released from the parked position when moved proximally by the actuator member when the latter is moved proximally by the needle unit shield (see below).

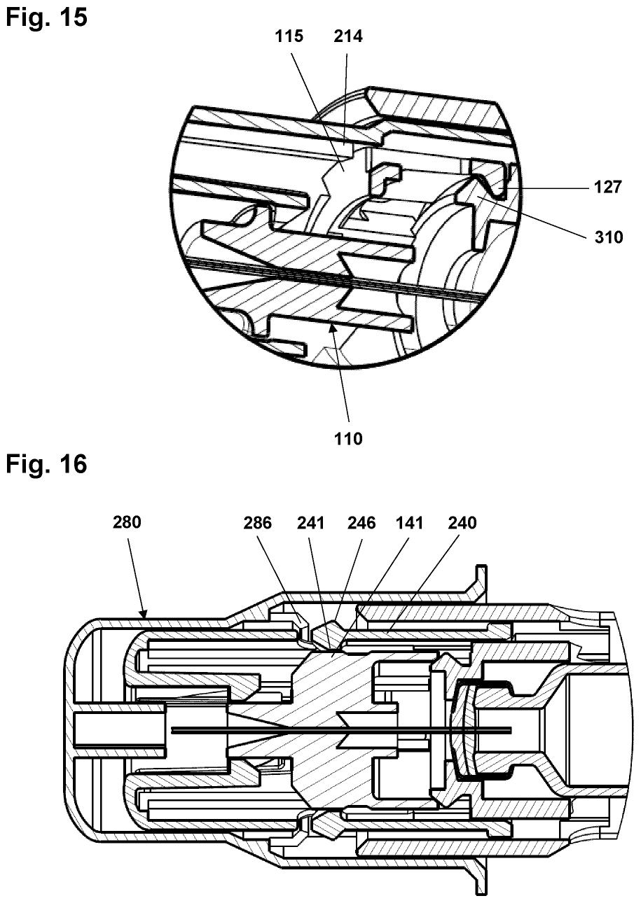

FIG. 15 shows in a detail view how the drive spring proximal hook 892 engages a longitudinally extending free spring edge 932 in axial sliding engagement. FIG. 16 shows in a detail view how the drive spring distal hook 891 has been rotated into engagement with the catch opening 821. FIG. 17 shows in a detail view how a drop lock finger 521 engages the housing drop lock surface 407 to prevent accidental axial movement of the actuator. The figure also shows the piston rod 650 engaging the cartridge piston 392 via the piston washer 395.

The shown embodiment may be assembled in the following steps: (i) The drive nut 600 is inserted in the housing snapping in place with an axial play, (ii) the control member 700 is inserted in the housing tower with the stop edges aligned, (iii) the actuator 500 is inserted with the legs arranged in the housing cartridge portion and with the cylindrical proximal portion 501 surrounding the control member and housing tower, shoulders on the housing inner wall serving to deflect the legs inwardly allowing them to be moved into the cartridge portion, (iv) the drive member drive tube 802 is inserted in the housing tower, (v) the return spring 590 is inserted distally engaging the actuator spring support flange 510 and proximally temporarily being held in place by the housing snap lock finger hooks 421, (vi) the piston rod 650 is inserted in the drive tube which is turned 180 degrees back and forth to allow the piston rod thread to engage the drive nut thread, (vii) the drive spring 890 is inserted in the spring housing 801 with the drive spring distal hook being rotated to be received in the catch opening 821, (viii) the spring base 900 is inserted partly into the housing and rotated to strain the drive spring, whereby during initial rotation one of the spring edges 932 engages the drive spring proximal hook 892, (ix) the spring base is subsequently moved fully into the housing whereby it snaps into engagement with the housing snap lock finger hooks 421 thereby displacing the return spring which then becomes seated against the spring base, at the same time the clicker finger 825 engages the ratchet surface 915, (x) the cartridge 390 is inserted into the cartridge holder and the piston washer is placed onto the cartridge piston, (xi) the housing cartridge portion is slightly ovalized allowing the cartridge holder to be inserted with the window walls 316 snapping into engagement with housing windows 416 while at the same time the flexible cartridge support arms 320 engage the pressure edges 437 and are deformed into the free space 322 distally of the arms to thereby eliminate axial play of the mounted cartridge. At the same time the piston washer 395 engages the piston rod distal end whereby the piston rod and the drive nut is moved slightly proximally with no gap being formed between the piston rod and the piston washer, and (xii) the drive nut is fused to the housing by application of e.g. a laser beam through housing nut openings 406. As a final step a label (not shown) is applied to the housing proximal portion to cover the snap locks and the nut openings. In an alternative embodiment a modified piston washer is snapped onto a modified piston rod before the cartridge holder is inserted into the housing, this preventing the piston washer from being dislocated before it is engaged by the piston rod.

Before turning to a description of the needle unit, operation of the drug delivery device (when actuated by a needle unit) will be described below with reference to FIGS. 18A-18F. As the drug delivery device can only be released to expel a dose of drug via a mounted needle unit the two units in combination can be said to form a combined drug delivery system.

When a needle unit is mounted on the cartridge mount 310 the actuator legs 502 are engaged by the needle unit hub 100 and shield 200 whereby the actuator is rotated (here: 20 degrees) from the initial lock position to an actuated un-locked position in which (i) the drop lock fingers 521 are no longer aligned with the housing drop lock surfaces 407, this allowing the actuator to subsequently be moved proximally, (ii) the PONR wedges 530 are aligned with the housing PONR flexible arms 408, and (iii) the anti-rotational clutch 517 is moved from its initial to its actuated position, the latter being described in greater detail below in connection with mounting of the needle unit.

More specifically, FIG. 18A shows (in part) the device in its pre-dosing state after a needle unit has been mounted. The control member 700 is positioned in its distal-most position with the stop edge 714 engaging the tower stop edge 434, this preventing the drive spring from rotating the drive tube 802 as the control member and the drive tube are in splined engagement. In addition (not shown) the second stop edge 729 engages tower stop knob 439. The actuation knob 516 has been rotated into alignment with the release flange 716.

When the user inserts the needle of the needle unit subcutaneously (see below), the actuator is moved proximally by the needle shield against the force of the return spring, the actuator actuation knob 516 thereby moving over the stop edge 714 and into engagement with the control member release flange 716 distal end. As the drop lock flange 511 moves with the actuator knob 516 the control member can be moved proximally. When the needle shield and thus the control member have been moved to their proximal-most position the control member stop edge 714 has been moved out of axial engagement with the tower stop edge 434 (see FIG. 18B), this allowing the control member and thereto splined drive tube 802 to rotate, this resulting in rotation and distal movement of the piston rod and thereby expelling of drug. During rotational movement of the drive member a clicking sound is produced as the clicker finger 825 is rotated relative to the ratchet surface 915.

Before the expelling mechanism is released the actuator PONR wedges 530 have been moved over the housing PONR stop surfaces (allowed by the flexibility of PONR flexible arms 408), this preventing the actuator, and thus the needle shield, from being returned to their initial position. Correspondingly, should the user pull out the inserted needle at this point, the non-returned needle shield will indicate to the user that actuation of the device is “in progress” and that an injection should be performed.

When the control member 700 starts to rotate, the helical flange 710 is moved into the gap between the tower helical surface end surface 433 and the distal end of the actuation knob 516, the three structures forming a threaded connection which assures that the control member is moved distally as it rotates (see FIG. 18C).

FIG. 18D shows the state just prior to end-of-dose. The control member stop edge 514 approaches engagement with the tower stop edge 434 and the control member release flange 716 approaches the actuation knob 516. Ultimately the two stop edges engage each other, and rotation and out-dosing come to an end-state. At the same time the release flange 716 has rotated the actuation knob 516 and thus the connector back to its initial rotational lock position (see FIG. 18E).

It should be noted that at this final state the actuation knob 516 is aligned with the flange cut-out 711 which would allow the control member to move proximally, e.g. by gravity if the device was used in an upside-down orientation, this moving the stop edge 714 axially out of engagement with the tower stop surface 434 thereby allowing the control member 700 to continue rotating and thus a further dose to be expelled. To prevent such a gravity-induced situation from arising, the splined engagement 730, 830 between the control member and the drive tube may be provided with friction structures, the friction being easily overcome by the energy provided by the drive spring but not by gravity. Alternatively, the control member may comprise an integrated return spring or an additional helical spring may be position between the two components.

When the user withdraws the needle from the skin, the actuation knob 516 is aligned with the flange cut-out 711 allowing the return spring to move the actuator (see FIG. 18F) and thereby also the needle shield distally to their initial distal-most positions. During this axial movement the actuator drop lock fingers 521 are rotationally aligned with the housing drop lock surfaces 407, however, due to their flexibility they will pass over the drop lock surfaces. Oppositely, during the final rotation of the actuator, the PONR wedges 530 are moved out of rotational alignment with the housing PONR stop surfaces 409. As the actuator is moved distally the drop lock flange 511 (hidden in FIG. 18F, see FIG. 18A) re-engages the control member flange 710 thereby moving the control member back to its initial distal position (see FIG. 18F) with the pointed catch 715 seated in the tower notch 435, and the serrated clutch portions 517 reengage the housing to re-activate the anti-rotational clutch in its initial rotational position.

At this state the control member 300, the actuator 500, and the drive member 800 have returned to their initial positions, whereas the piston rod has been moved distally corresponding to the pre-set dose size and the drive spring has been de-strained corresponding to a single dose. When the number of doses for which the device is designed, e.g. 4 doses, have been expelled the piston rod has been moved to its distal-most position in which the piston rod stop surface 655 is positioned in close proximity to control member stop flanges 735, this preventing the control member to be further actuated.

As will be described below for the needle unit, in this state the needle shield has been locked in its distal position and thus prevents the dose engine to be released, this providing protection against double-dosing. To allow a further dose to be expelled a new needle unit has to be mounted.

As seen e.g. in FIG. 6A, the housing distal end comprises an indicator opening 405. When the needle unit hub member 100 is rotated during actuation an indicator portion on the hub member moves into alignment with the indicator opening, this indicating that a dose of drug has been expelled.

Having described in detail the components and operation of the drug delivery device per se, in the following the needle unit and its operation will be described in detail.

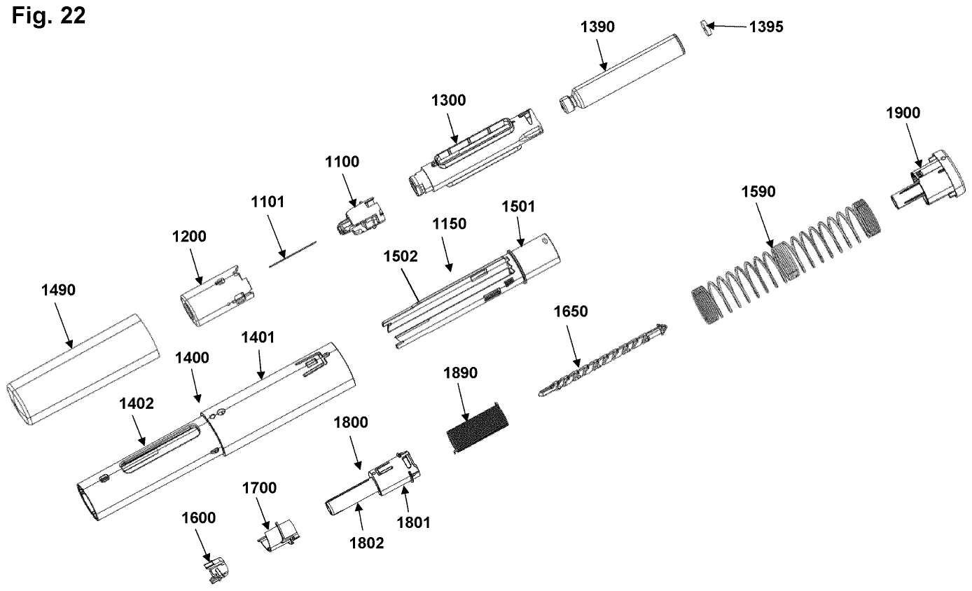

As shown in FIGS. 1 and 3 the drug delivery device 1 is adapted to receive a shielded needle unit 2, comprising a needle hub 100 adapted to be mounted on a corresponding mount on the drug delivery device, a subcutaneous hollow needle 101 mounted in the hub and comprising a pointed free distal end portion adapted to be inserted subcutaneously through the skin of a user and a pointed free proximal end portion adapted to be penetrate a pierceable drug cartridge septum, as well as a shield member 200 in which the needle hub is arranged. The subcutaneous hollow needle 200 comprises bevelled proximal and distal ends and is arranged in a hub bore and secured in place by e.g. adhesive. The needle unit is supplied as a needle assembly further comprising a container 280 (see FIG. 22) adapted to receive the needle unit in an assembled state, the container having an open end adapted to be sealed with a flexible foil member thereby providing a sealed, sterile interior for the needle unit when supplied to the user.

### Needle Unit

FIG. 1 shows a needle unit 2 mounted on an injection device 1 adapted to releasably receive the needle unit. In the shown embodiment the shield has an outer super-elliptic cross-section configuration allowing it to be received in the correspondingly formed distal opening of the injection device 3. Alternatively other non-circular designs could be implemented, e.g. square or triangular, or the needle unit could have a circular design with rotational orientation being provided by cooperating guide structures.

As will be apparent from the below detailed description of an exemplary embodiment of a needle unit the functionality of the unit relies on rotational movement between the hub and the shield, this irrespective of the outer configuration of the shield.

As will be explained in greater detail below, the needle hub 100 (in the following also just “hub” or “hub member”) and the shield member 200 (in the following also just “shield”) comprise a number of interacting structures allowing the shield and hub to move axially and rotatably relative to each other in a controlled manner during use and operation of the needle unit. In the below-described embodiment the shield is rotationally locked relative to the cartridge mount and the hub is axially locked relative to the cartridge mount when the needle unit is mounted on the cartridge mount. Rotational movement of the hub is controlled by axial movement of the shield relative to the cartridge mount and thus the hub.

As will also be explained in greater detail below, the container and the shield member comprise interacting structures allowing the container to be used as a mounting and removal tool for the needle assembly during use in an efficient and user-friendly manner. The shield, hub and container generally comprise functional structures in opposed pairs, however, any suitable numbers of such structures could be used, e.g. one, two or three.

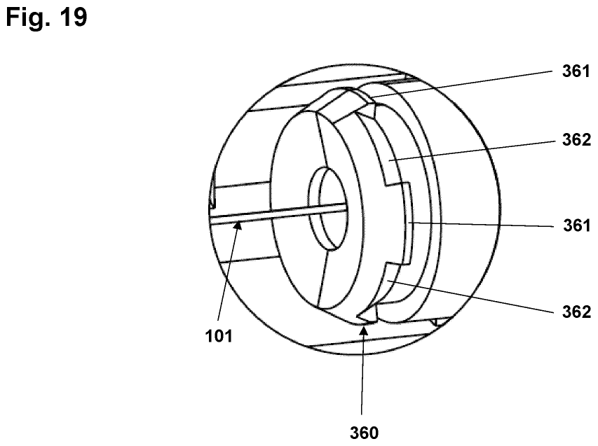

As shown in FIGS. 19A and 19B the shield 200 has a generally tubular configuration with a circumferential outer wall 210, a proximal opening with a circumferential edge 211, and a distal end surface 201 with a smaller distal opening 212 from which a tower structure protrudes axially inwardly. The tower structure comprises a circumferential skirt portion 215 from which first and second pairs of opposed arms extend proximally with a rotational offset of 90 degrees in the shown embodiment. Between the arms the skirt portion comprises free grip edge portions 216. The first pair of longer arms is in the form of flexible stop arms 220 each having a hook portion 225 at the proximal free end with a distally facing axial stop surface 221 adapted to engage corresponding proximally facing stop surfaces 121 on the hub tower portion (see below) as well as a proximally facing ramp surface 222 used during assembling of the needle unit. The second pair of shorter arms is in the form of flexible control arms 230 each having a hook portion 235 at the proximal free end with a proximally facing ramp surface 231 and a distally facing control surface 232, the surfaces being adapted to engage corresponding distally facing ramp surfaces respectively proximally facing control surfaces on the hub tower portion (see below). At the proximal end the shield comprises opposed pairs of inner actuation ribs 213 adapted to engage corresponding flexible hub arms (see below). The actuation ribs 213 also serve to lock the coupling arms (see below) and to centre the generally circular hub in the super-elliptic shield and thus assure stability during axial and rotational movement between the hub and shield during operation. The shield further comprises a pair of opposed locking ribs 217 on the shield inner wall surface, each rib having a proximally facing locking surface 218 adapted to engage a corresponding locking surface on the hub. The locking ribs proximally extend into lower torque ribs 219 adapted to engage torque flanges on the hub (see below). The shield wall 210 is further provided with an outer pair of opposed mounting ribs 245 adapted to engage corresponding shield slots 415 in the housing, a pair of opposed windows 240 adapted to allow outwards movement of the hub coupling arms (see below), as well as a first indicator opening 241 and a second indicator opening 246. In the shown embodiment the first indicator opening 241 is “open” as for design reasons the shield edge 211 comprises cut-outs for structures in the drug delivery device.

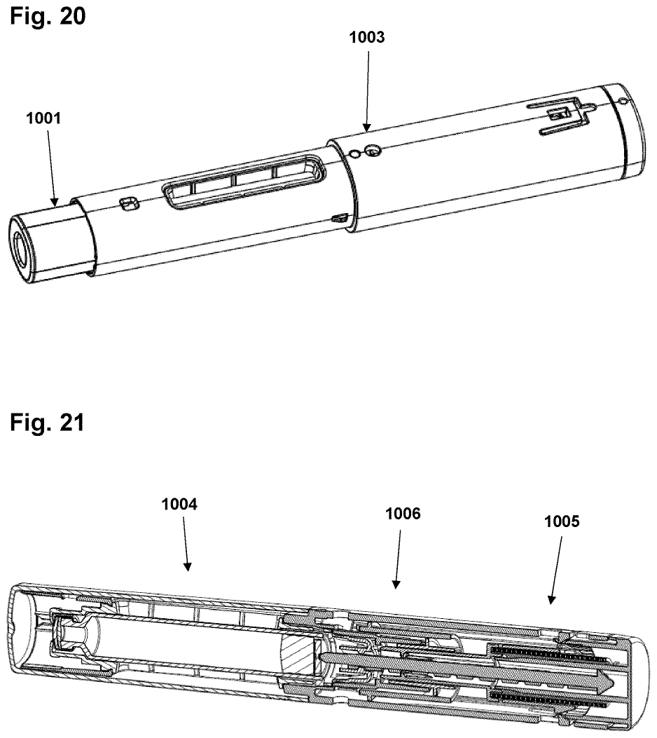

As shown in FIGS. 20A and 20B the hub 100 comprises a distal tower portion 110 comprising a central bore 111 adapted to receive a subcutaneous needle as well as a proximal skirt portion 120. At the distal end the tower portion comprises three pairs of opposed function surfaces adapted to cooperate with corresponding surfaces on the shield: (i) a pair of proximally facing stop surfaces 121 adapted to engage the distally facing stop surfaces 221 on the shield, (ii) a pair of distally facing ramp surfaces 131 adapted to engage the proximally facing ramp surfaces 231 on the shield, and (iii) a pair of inclined proximally facing control surfaces 132 adapted to engage the distally facing control surfaces 232 on the shield during operation. At the proximal end the tower portion comprises a pair of opposed snap indentations 135 adapted to engage the control arm hook portions 235. The skirt portion 120 comprises an opposed pair of proximally extending flexible coupling arms 126, each arm having an outer surface 123 adapted to engage the shield inner actuation ribs 213 during operation, and an inwardly facing snap coupling ridge 127 arranged at the free proximal end of the coupling arm and adapted to engage a corresponding coupling structure on the cartridge mount 310. The skirt portion further comprises a pair of distally facing lock surfaces 118 adapted to engage the proximally facing shield locking surfaces 218 during operation. The skirt portion further comprises a pair of radially protruding opposed drop lock release flanges 114 adapted to engage the actuator leg release surfaces 524, a pair of radially protruding opposed torque flanges 119 adapted to engage the shield torque ribs 219, as well as a pair of opposed indicator cut-outs 115.

As shown in FIGS. 21A and 21B the container 280 has a generally tubular configuration with a super-elliptic circumferential outer wall 281, a proximal opening with a circumferential flange 282 and a closed distal end 283 from which a tower structure 285 and a pair of opposed snap lock fingers 290 extend axially inwardly. The snap lock fingers each comprises an outwardly oriented snap protrusion 296 adapted to releasably engage the shield tower grip edge portions 216 to provide a snap coupling. Distally the container further comprises a plurality of inner support ribs 286 adapted to engage the shield outer surface and support the shield when arranged in the container. With the shield mounted in the container the proximal portion provides a circumferential space 299 (see FIG. 22) between the container and the shield allowing the correspondingly shaped drug delivery housing portion to be received therein during mounting of the needle unit on the drug delivery device.

During assembly the hollow subcutaneous needle 101 with bevelled proximal and distal ends is arranged in the hub bore and secured in place, e.g. by means of adhesive, this providing a free distal end portion 102 and a free proximal end portion 103. The hub 100 is subsequently inserted in the shield 200 with the stop surfaces 121 and the ramp surfaces 131 rotationally aligned with the shield stop surfaces 221 respectively the shield ramp surfaces 231, this allowing the shield stop surfaces to snap into engagement with the hub stop surfaces 121. Also the hub coupling arms 126 are rotationally aligned with the shield actuation ribs 213. The assembled needle unit is then inserted into the container with the container snap lock fingers 290 snapping into engagement with the shield tower grip edge portions 216. As seen in FIG. 22 an axial gap is provided between the container tower proximal end and the hub tower distal end. As a final assemble step a flexible foil member (not shown) is attached to the container proximal flange 282 thereby sealing the interior for subsequent sterilization. FIG. 22 shows in cross-section the needle assembly with the needle unit positioned in the container before the seal foil is attached.

In the following the different features and aspects of the above-described needle unit and container combination will be described with reference to figs. X1-X12 showing a needle unit being mounted on a corresponding drug delivery device, operated to allow an amount of fluid drug to be injected subcutaneously and subsequently removed from the drug delivery device.

After the flexible seal foil has been removed from the container 280 by the user, the container is to be used as a tool to mount the needle unit on the drug delivery device 1 with a corresponding cartridge mount 310, see fig. X1. In the shown embodiment the cartridge mount is arranged proximally of the distal end of the drug delivery device housing 400 with a circumferential space between the cartridge holder 300 and the housing adapted to receive the shield proximal portion in non-rotational engagement by the cooperating mounting ribs 245 and shield slots 415. In the shown embodiment the non-circular design makes it easy for the user to orient the needle assembly rotationally correct relative to the drug delivery device in either of its two possible rotational positions.

Initially the locked hub coupling arms 126 engage the cartridge mount coupling flange portions 311 this allowing the shield to be pushed forward by the container to an axial position in which the shield actuation ribs 213 are not engaging the flexible hub arm outer surfaces 123. During this movement the gap provided between the container tower proximal end and the hub tower distal end is eliminated allowing the container tower to push directly on the hub.

The container thus assures that the shield and hub can be pushed firmly into engagement with the cartridge mount by the user, this allowing the free proximal needle end portion 103 to penetrate the cartridge septum and the flexible hub coupling arms 126 to be initially moved radially outwards in the receiving shield windows 240 and subsequently snap radially inwards into engagement with the corresponding snap coupling flanges 311 on the cartridge mount, see fig. X2.

During the axial coupling movement of the needle unit the hub drop lock release flanges 114 engage the inclined leg release surfaces 524 on the spring-biased actuator. Initially the actuator is moved axially until the anti-rotational clutch portions 517 are moved out of engagement with the housing, this allowing the actuator to be rotated by the axial movement of the hub. To counter the torque exerted on the hub during rotation of the actuator, the hub is supported by the shield (which is non-rotationally coupled to the housing 400) via the torque flanges 119 engaging the torque ribs 219. Subsequently the shield actuator ribs 213 engage the leg actuation surfaces 523 of the actuator 500 and moves axially together with the drop lock release flanges 114. Depending on the actual design of the different components, the actuator may be fully rotated (here: 20 degrees) during mounting of the needle unit. Alternatively, final rotation of the actuator may take place when the actuator subsequently is allowed to be moved distally by the return spring 590.

Axial mounting movement of the needle unit stops when the hub engages the cartridge mount, this indicating to the user that the needle unit has been mounted on the cartridge hub. When the user stops pushing on the container (or starts to pull the container away) the spring-biased actuator legs 502 will push the shield 200 slightly distally until the distally facing axial stop surfaces 221 on the shield tower engage the corresponding proximally facing stop surfaces 121 on the hub tower portion. At the same time the clutch portions 517 reengage with the housing lock ribs 447 in the actuated rotational position. As the shield is moved distally the shield actuation ribs 213 will move into engagement with the flexible hub arm outer surfaces 123 and thereby prevent radial outwards movement thereof, this securely locking the hub 100 to the cartridge mount 310 corresponding to an actuated hub coupling lock state in which a mounted needle unit cannot be removed from the drug delivery device, see fig. X3.

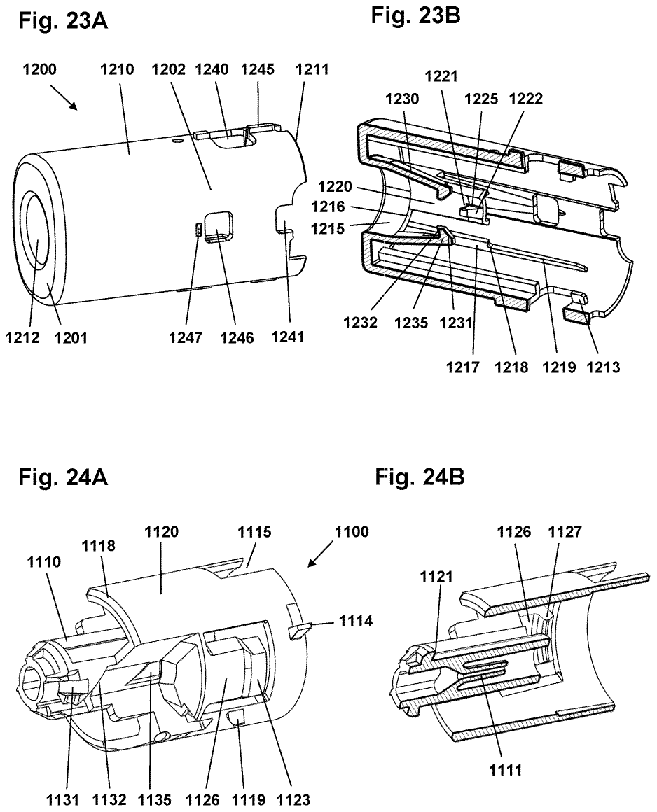

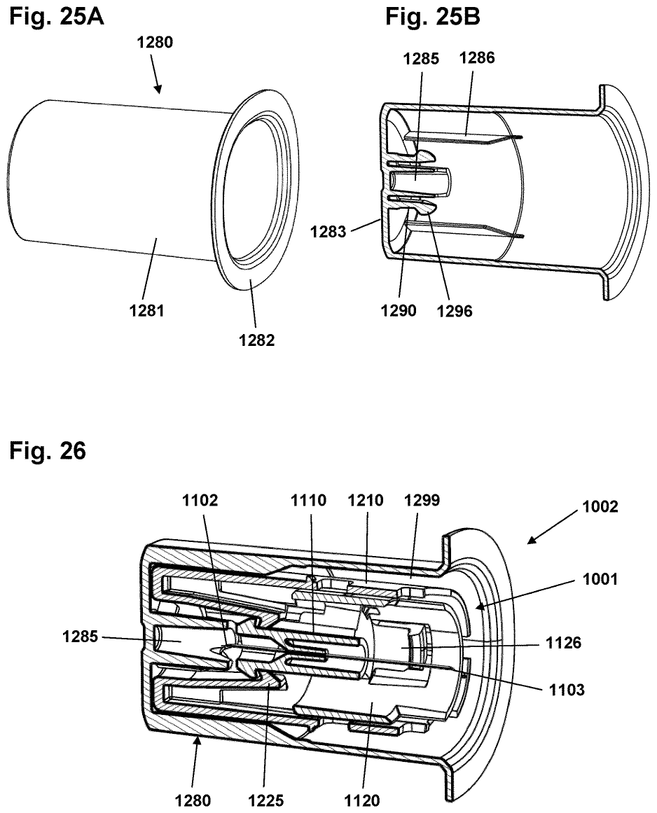

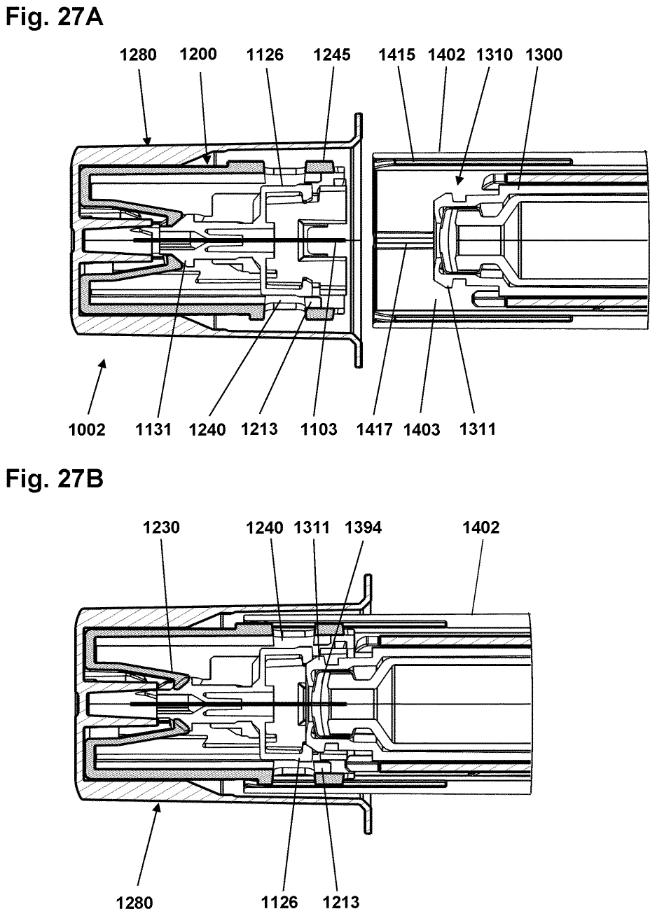

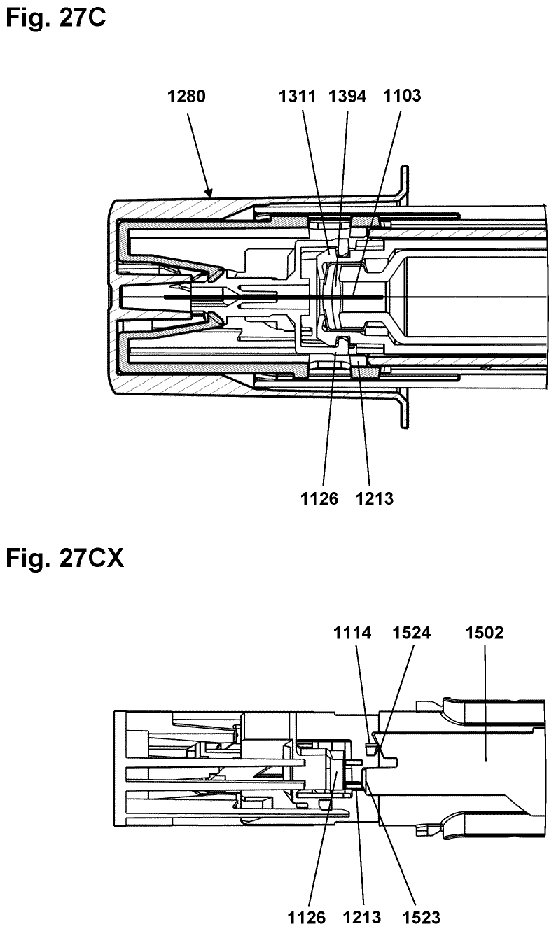

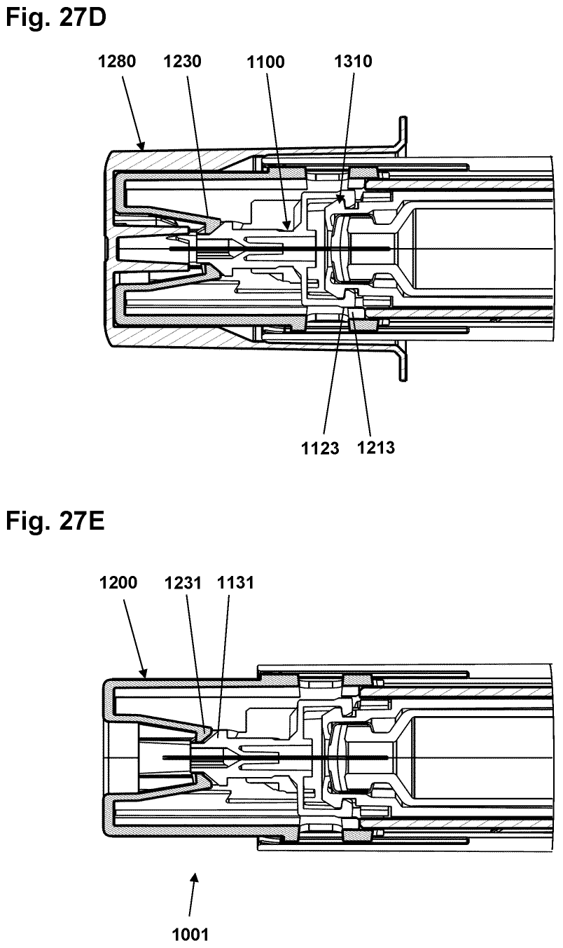

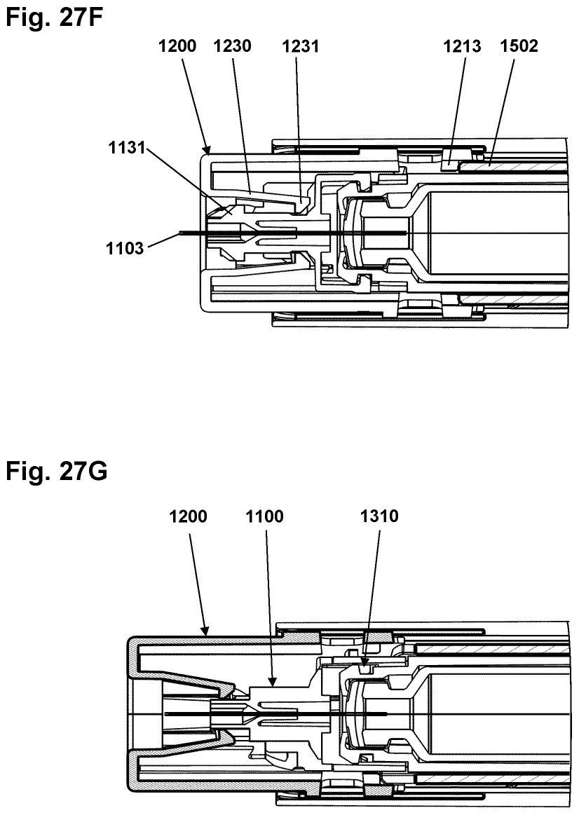

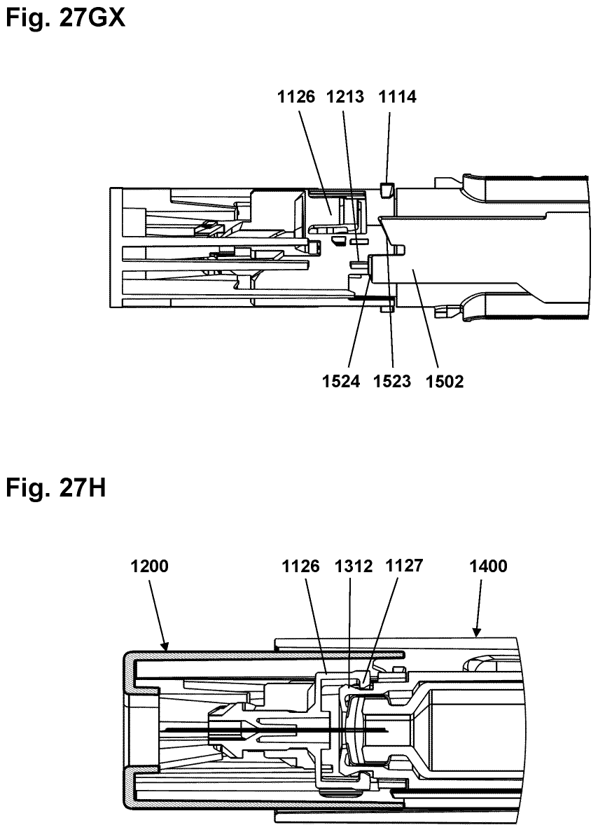

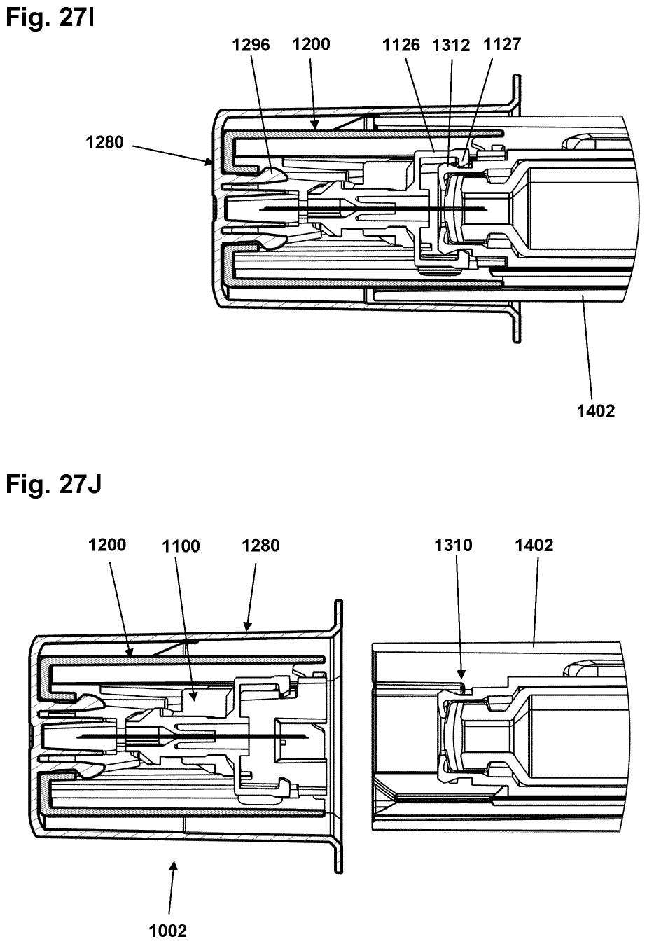

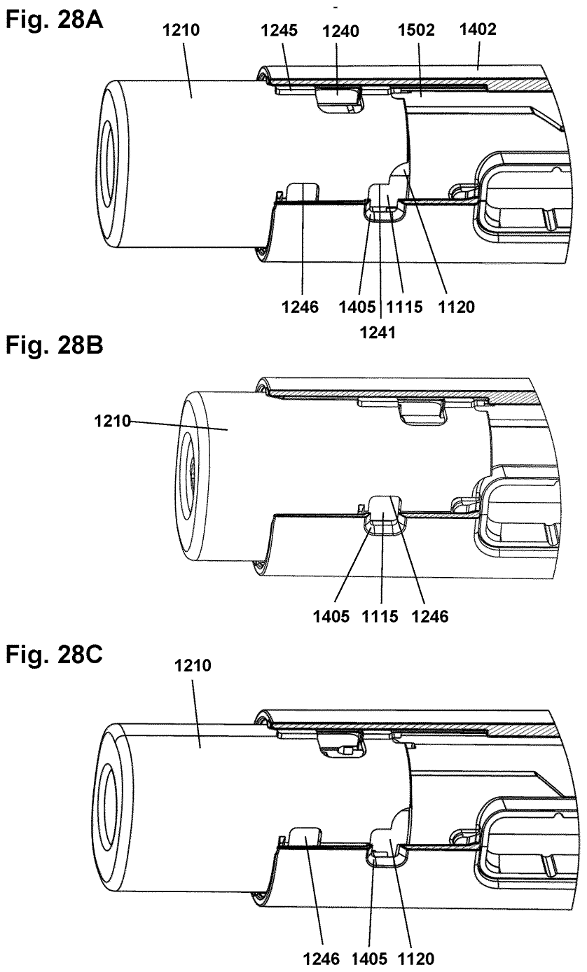

As the user pulls the container 280 further distally to fully remove it, the container snap coupling 296 will disengage the shield 200. When the container is fully removed the drug delivery device with the mounted needle unit is ready for use as shown in fig. X5. In this state the housing indicator opening 405 is aligned with a shield first indicator opening 241 and a hub indicator cut-out 115. The hub skirt 120 is thus not visible to the user (see FIG. 23A).

When the user pushes the needle unit towards a skin surface the shield 200 is pushed proximally allowing the needle distal end 103 to be inserted subcutaneously. During initial proximal movement of the shield the ramp surfaces 231 on the flexible control arms 230 are pushed over the hub tower ramp surfaces 131. As the shield is moved further proximally to its fully retracted position the shield actuator ribs 213 pushes the pair of actuator legs 502 proximally to thereby release the drug delivery device expelling mechanism thereby starting subcutaneous injection as described above (see figs X6). As appears, the actuator legs 502 serve both as a locking actuator for the hub coupling and as release members for the expelling mechanism. During out-dosing the shield is held in its fully retracted position by the snap lock 135, 235.

After the clicking sound stops and the dose thus has been fully expelled the user withdraws the needle unit from the skin surface thereby allowing the spring-biased actuator legs 502 to move the shield 200 distally to its fully extended position again covering the needle distal portion 103. During this movement the control surfaces 232 on the control arms 230 will engage the inclined proximally facing control surfaces 132 on the hub tower, which will force the needle hub 100 to rotate as the shield is rotationally locked to the drug delivery device, see fig. X7. In the shown embodiment the hub is rotated 45 degrees relative to the shield.

During rotation of the hub relative to the shield a number of structures are moved into and out of engagement with each other:
(i) As the hub rotates, a portion of the hub skirt 120 neighbouring the hub cut-out 115 aligned with the housing indicator opening 405 will rotate into alignment with the opening and thus be visible to the user (e.g. by having a contrasting colour), this indicating that the needle unit has been used and correspondingly that a dose of drug has been expelled. During drug expelling with the shield in its retracted position the shield second indicator opening 246 is aligned with the housing indicator opening 405.
(ii) As the hub rotates, the flexible hub coupling arms 126 rotate out of engagement with the shield actuation ribs 213 thereby allowing the coupling arms to move radially outwards, see fig. X8, and thus the hub 100 to be removed from the cartridge mount 310. To reduce the force necessary to disengage the hub fingers from the cartridge mount, the hub fingers are rotated to a position on the cartridge mount having lower and less inclined release flange portions 312.
(iii) As the needle is fixed in the hub it follows that the needle will rotate with the rotating hub, however, it may not be desirable that the needle rotates when fully inserted subcutaneously. Correspondingly, the hub and shield can be designed with an axial “play” before the shield control surfaces 232 engages the inclined hub control surfaces 132, this allowing the needle to be at least partly withdrawn from the skin before rotation starts. Indeed, the later rotation starts the steeper the inclination of the hub control surfaces have to be.
(iv) Before operation of the needle unit, the pair of locking ribs 217 on the shield inner surface were free to move in the axial direction thus allowing the shield to be moved from its extended to its retracted position. As the hub is rotated the pair of distally facing locking surfaces 118 are rotated into alignment with the proximal ends 218 of the locking ribs 217 thereby preventing repeated retraction of the shield and thus use of the needle unit, thereby providing a safety lock, see figs. X9. Correspondingly, the locking surfaces should be designed to withstand relatively large forces to prevent re-activation of the needle unit, e.g. if the pen device is dropped on a hard surface or in a mis-use scenario. Further, in case the drug delivery device as in the present embodiment is shield released the shield lock will also serve as a double dose prevention means.
(v) In the shown embodiment the container is intended to be used as a tool also for removing the needle unit, see fig. X10. When the user re-attaches the container, the container snap coupling 296 will engage the shield 200. The container snap coupling is designed to have a release force larger than the release force necessary to pull the flexible hub coupling arms 126 axially out of engagement with the cartridge mount release flanges 312, this allowing the needle unit to be removed from the cartridge mount securely held in the container after which it can be safely discarded, see fig. X12. As the needle unit is locked to the container via the container snap coupling and the proximal end is positioned a certain distance inside the container and only being surrounded by a small free space, removal of the needle unit from the container would be difficult.
(vi) As the hub rotates, the drop lock release flanges 114 are rotated out of alignment with the actuator leg release surfaces 524, this preventing that a used and locked needle unit can be used to release the actuator drop lock.

### Alternative Embodiments

In the above-described embodiment, when the hub rotates relative to the cartridge mount the flexible coupling arms 126 are rotated to align with the inclined release flanges 312 allowing the hub coupling arms to disengage with ease which may encourage the user to remove the needle unit without using the container. To encourage the user to use the container, the release flanges may be modified to require a larger release force which would make it more difficult to merely grab and pull the shield out of engagement with the hub mount. To allow this the snap coupling between the container and the shield has to be able to transfer the required force, however, as the same snap coupling should be designed to allow for easy removal of the container after initial mounting of the needle unit this may not be desirable.

Correspondingly, a needle assembly may be provided comprising a snap coupling between the container and the shield, the snap coupling being actuatable between a first state in which the needle unit can be removed from the container using a first amount of force, and a second state in which the needle unit can be removed from the container using a second higher amount of force. The assembly may be operated between the two states by the rotational movement of the hub inside the shield. A detailed description of such an arrangement is described in co-pending application EP 23174814.6 which is hereby incorporated by reference.

In the above-described embodiment an indicator is incorporated in the drug delivery housing and being operated by the rotational movement of the hub. Although indicator actuation is controlled by the needle unit, the placement of the indicator window 405 on the housing is designed to associate the indicator with operation of the device per se and thus indicate that a dose of drug has been expelled which will be the case when the shield has been returned to its extended and now locked position.

However, it may be desirable to provide the indicator on the shield to instead directly indicate that the needle module has been used and is now locked. Correspondingly, instead of the device housing the shield may be provided with an indicator window and the hub may be provided with an indicator surface which initially is not aligned with the window but moved into alignment therewith when the hub is rotated after use.

In the above description of exemplary embodiments, the different structures and means providing the described functionality for the different components have been described to a degree to which the concept of the present invention will be apparent to the skilled reader. The detailed construction and specification for the different components are considered the object of a nor-mal design procedure performed by the skilled person along the lines set out in the present specification.

1. A drug delivery assembly comprising a drug delivery device and a thereon mountable needle unit, the needle unit comprising:
a needle hub,
a hollow needle mounted in the needle hub and having a pointed distal end protruding from the needle hub, the hollow needle defining a reference axis,
a shield in which the needle hub is arranged, the shield being axially moveable relative to the needle hub between an extended position in which the shield axially covers the needle distal end, and a retracted position in which the needle distal end protrudes from the shield, the shield comprising a proximal skirt portion, and
coupling structure forming part of the needle hub and allowing the needle unit to be mounted on the drug delivery device,
wherein the needle hub is (a) arranged fully within the shield or (b) the needle hub extends from the proximal end of the shield skirt portion less than the distance between the needle distal end and the shield distal end with the shield in its extended position,
the drug delivery device comprising:
a cartridge mount allowing the coupling structure to be mounted on the drug delivery device, and
a distal housing wall portion surrounding the cartridge mount and forming a circumferential space adapted to receive the shield skirt portion
wherein:
the circumferential space and the shield skirt portion have corresponding non-circular configurations allowing the shield skirt portion to be axially received in the circumferential space in one or more pre-determined rotational mounting positions, and
the coupling structure and the cartridge mount are configured to allow the coupling structure to be mounted on the drug delivery device by axial engagement in any of the pre-determined rotational positions.

2. The drug delivery assembly as in claim 1, wherein the non-circular configuration is one of:
a non-symmetrical form providing one rotational mounting position,
an oval or rectangular form providing two rotational mounting positions,
a triangular form providing three rotational mounting positions, or
a square form providing four rotational mounting positions.

3. The drug delivery assembly as in claim 1, wherein the shield and the drug delivery device comprise corresponding axial guide structures.

4. The drug delivery assembly as in claim 1, wherein the coupling structure is actuatable between:
an initial mounting state in which the needle unit can be mounted on the drug delivery device, and
a lock state in which the mounted needle unit cannot be removed from the drug delivery device,
wherein the coupling structure is actuated from the initial mounting state to the lock state when the needle unit is mounted on the drug delivery device.

5. The drug delivery assembly as in claim 4, wherein;
the coupling structure is actuated from the mounting state to the lock state by axial movement of the shield relative to the needle hub from an initial mounting position to an actuated lock position.

6. The drug delivery assembly as in claim 5, wherein the shield actuated lock position corresponds to the extended position.

7. The drug delivery assembly as in claim 1, wherein the coupling structure comprises:
deflectable gripping fingers forming part of the needle hub and being adapted to grip a corresponding coupling structure on the drug delivery device, and
blocking structure forming part of the shield and being adapted to block radial movement of the deflectable gripping fingers corresponding to the lock state in which a mounted needle unit cannot be removed from the drug delivery device,
wherein the deflectable gripping fingers and the blocking structure are moveable relative to each other from an initial mounting position in which radial movement of the gripping fingers is possible, to an actuated lock position in which radial movement of the gripping fingers is blocked, this preventing the needle unit from being removed from the drug delivery device.

8. The drug delivery assembly as in claim 4, the coupling structure further being actuatable between:
the lock state, and
an actuated release state in which the needle unit can be removed from the drug delivery device.

9. The drug delivery assembly as in claim 8, wherein:
the coupling structure is actuated from the lock state to the release state when the shield is moved from its retracted to its extended position.

10. The drug delivery assembly as in claim 8, wherein:
the shield is mounted non-rotatable on the drug delivery device.

11. The drug delivery assembly as in claim 10, wherein:
the coupling structure is actuated from the lock state to the release state by rotational movement of the needle hub relative to the shield.

12. The drug delivery assembly as in claim 1, wherein:
when the shield is moved from the retracted to the extended position, the shield and the needle hub move relative to each other to a locking position in which the shield cannot be retracted.

13. A needle unit adapted to be releasably mounted on a drug delivery device, the drug delivery device comprising:
a cartridge mount, and
a housing wall portion surrounding the cartridge mount and forming a circumferential space having a non-circular configuration,
the needle unit comprising:
a needle hub,
a hollow needle mounted in the needle hub and having a pointed distal end protruding from the needle hub, the hollow needle defining a reference axis,
a shield in which the needle hub is arranged, the shield being axially moveable relative to the needle hub between an extended position in which the shield axially covers the needle distal end, and a retracted position in which the needle distal end protrudes from the shield, the shield comprising a proximal skirt portion, and
coupling structure forming part of the needle hub and allowing the needle unit to be mounted on the drug delivery device,
wherein the needle hub is (a) arranged fully within the shield or (b) the needle hub extends from the proximal end of the shield skirt portion less than the distance between the needle distal end and the shield distal end with the shield in its extended position,
wherein the shield comprises a proximal skirt portion having a non-circular configuration allowing the shield skirt portion to be axially received in a correspondingly configured circumferential space on the drug delivery device.

14. The needle unit as in claim 13, wherein the non-circular configuration is one of:
a non-symmetrical form,
an oval or rectangular form,
a triangular form, or
a square form.

15. The needle unit as in claim 14, wherein the shield proximal skirt portion comprise outer axial guide structures.
